SEQUENCE LISTING (3 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKANVDVLTKAKSQ

SEQ ID NO: 2 - protein, 34 aa, Homo sapiens
SVSEIQLMHNLGKHLNSMERVEWLRKKLQDVHNF

SEQ ID NO: 3 - protein, 31 aa, Homo sapiens
SVSEIQLMHNLGKHLNSMERVEWLRKKLQDV

(19) United States
(12) Patent Application Publication
(10) Pub. No.: US 2007/0173447 A1
(43) Pub. Date: Jul. 26, 2007

(54) METHOD FOR TREATING OSTEOPOROSIS BY INTRANASAL DELIVERY OF TERIPARATIDE WITH AN ANTI-RESORPTIVE AGENT

(75) Inventor: Steven C. Quay, Seattle, WA (US)

Correspondence Address: NASTECH PHARMACEUTICAL COMPANY INC, 3830 MONTE VILLA PARKWAY, BOTHELL, WA, 98021-7266, US

(73) Assignee: Nastech Pharmaceutical Company Inc.

(21) Appl. No.: 11/552,810

(22) Filed: Oct. 25, 2006

Related U.S. Application Data
(60) Provisional application No. 60/730,547, filed on Oct. 25, 2005.

(51) Int. Cl. A61K 38/29

(52) U.S. Cl. 514/12

(57) ABSTRACT

A method for treating osteoporosis in a mammal by administering intranasally a therapeutically effective amount of a PTH formulation to the mammal in combination with administration of an anti-resorptive agent. The PTH formulation can contain teriparatide.

This application claims the benefit under 35 U.S.C. § 119(e) of U.S. Provisional Application No. 60/730,547, filed Oct. 25, 2005, which is hereby incorporated by reference in its entirety.

### BACKGROUND OF THE INVENTION

Osteoporosis is a systemic skeletal disease characterized by low bone mass and microarchitectural deterioration of bone tissue which results in increased bone fragility and susceptibility to fracture. It most commonly affects older populations, primarily postmenopausal women.

The prevalence of osteoporosis poses a serious health problem. The National Osteoporosis Foundation has estimated that 44 million people are experiencing the effects of osteoporosis or osteopenia. By the year 2010, osteoporosis will affect more than 52 million people and, by 2020, more than 61 million people. Women are affected in greater numbers than men are because men have a higher peak bone density. Furthermore, as women age the rate of bone turnover increases, resulting in accelerated bone loss because of the lack of estrogen after menopause.

One goal in the treatment of osteoporosis is to maintain or increase bone strength. This prevents fractures throughout the patient's life, and minimizes osteoporosis-related morbidity and mortality. The medications that have been used most commonly to treat osteoporosis include calcium, vitamin D, estrogen (with or without progestin), bisphonates, selective estrogen receptor modulators (SERMs), and calcitonin.

Parathyroid hormone (PTH) has recently emerged for treating osteoporosis. Unlike therapies that only reduce bone resorption, PTH increases bone mass which results in greater bone mineral density (BMD). PTH has multiple actions on bone, some direct and some indirect. PTH increases the rate of calcium release from bone into blood. The chronic effects of PTH are to increase the number of bone cells both osteoblasts and osteoclasts, and to increase the remodeling of bone. These effects are apparent within hours after PTH is administered and persist for hours after PTH is withdrawn. PTH administered to osteoporotic patients leads to a net stimulation of bone formation, especially in trabecular bone in the spine and hip, resulting in significant reduction in fractures. The bone formation is believed to occur by the stimulation of osteoblasts by PTH because osteoblasts have PTH receptors.

PTH is a secreted, 84 amino acid polypeptide having the amino acid sequence:

SEQ ID NO:1

Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-His-Asn-Phe Val Ala
Leu Gly Ala Pro Leu Ala Pro Arg Asp Ala Gly Ser
Gln Arg Pro Arg Lys Lys Gln Asp Asn Val Leu Val
Glu Ser His Glu Lys Ser Leu Gly Glu Ala Asp Lys
Ala Asn Val Asp Val Leu Thr Lys Ala Lys Ser Gln

Studies in humans with certain forms of PTH have demonstrated an anabolic effect on bone and have prompted significant interest in its use for the treatment of osteoporosis and related bone disorders.

For example, it has been demonstrated in humans that PTH₁₋₃₄, the N-terminal 34 amino acid sequence of the bovine and human hormone which has been deemed biologically equivalent to the full length hormone, enhances bone growth, particularly when administered in a pulsatile mode by subcutaneous injection. A slightly different fragment of PTH, human PTH₁₋₃₈, has shown similar results. PTH₁₋₃₄ has the amino acid sequence:

(SEQ ID NO:2)

Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-His-Asn-Phe

PTH preparations have been reconstituted from fresh or lyophilized hormone, and may incorporate various forms of carrier, excipient and vehicle. Most are prepared in water-based vehicles such as saline, or water acidified typically with acetic acid to solubilize the hormone. The majority of reported formulations also incorporate albumin as a stabilizer [see for example, Reeve, et al., Br. Med. J. 280:6228, 1980; Reeve, et al., Lancet 1:1035, 1976; Reeve, et al., Calcif. Tissue Res. 21:469, 1976; Hodsman, et al., Bone Miner 9(2):137, 1990; Tsai, et al., J. Clin. Endocrinol Metab. 69(5):1024, 1989; Isaac, et al., Horm. Metab. Res. 12(9):487, 1980; Law, et al., J. Clin Invest. 72(3):1106, 1983; and Hulter, J. Clin. Hypertens 2(4):360, 1986]. Other reported formulations have incorporated an excipient such as mannitol, which is present either with the lyophilized hormone or in the reconstitution vehicle.

PTH₁₋₃₄ (generic name: teriparatide, see WHO, Chronicle 37, No. 5, suppl., 1983) is currently marketed in the U.S. as FORTEO®, and in Europe as FORSTEO®, by Eli Lilly (Indianapolis, Ind.) for the treatment of postmenopausal women with osteoporosis who are at high risk of fracture. This drug is administered by a once daily subcutaneous injection of 20 μg in a solution containing acetate buffer, mannitol, and m-cresol in water at pH 4.

FORTEO® is manufactured by recombinant DNA technology using an Escherichia coli strain. PTH1-34 has a molecular weight of 4117.87 daltons. Reviews on PTH1-34 and its clinical that have been published, including, e.g., Brixen, et al., 2004; Dobnig, 2004; Eriksen and Robins, 2004; Quattrocchi and Kourlas, 2004, are hereby incorporated by reference. The safety of teriparatide has been evaluated in over 2800 patients in doses ranging from 5 to 100 μg per day in short term trials. Doses of up to 40 μg per day have been given for up to two years in long term trials. Adverse events associated with FORSTEO were usually mild and generally did not require discontinuation of therapy. The most commonly reported adverse effects were dizziness, leg cramps, nausea, vomiting and headache. Mild transient hypercalcemia has been reported with FORSTEO which is usually self limiting within 6 hours.

Currently FORTEO® is administered as a daily subcutaneous injection. The following Cmax and AUC values are described for various doses of FORTEO (20 ug is the commercially approved dose).

| SC Dose | | CL/F | AUC₀₋ₜ | Cₘₐₓ |
|---|---|---|---|---|
| (μg) | N | (L/hr) | (pg hr/ml) | (pg/ml) |
| 20 | 22 | 152.3 ± 91.2 | 165 ± 67.6 | 151.0 ± 56.9 |
| 40 | 16 | 124.3 ± 65.8 | 393 ± 161 | 256.2 ± 117.5 |
| 80 | 22 | 104.4 ± 27.9 | 816 ± 202.2 | 552.8 ± 183.6 |

Two classes of drugs are available for treating osteoporosis: antiresorptive drugs, such as bisphosphonates reduce bone resorption, while anabolic agents, such as teriparatide, primarily stimulate bone formation. The approval of teriparatide (FORTEO®) by the FDA for the treatment of osteoporosis was issued with the recommendation that therapy not last for more than two years. No recommendations were given for what to do after two years. Results in humans and animal models suggest that gains in bone mineral density achieved by teriparatide are lost if an antiresorptive agent is not administered after treatment.

Continuously elevated teriparatide actually increases bone turnover and can cause osteoporosis by stimulating bone resorption by osteoclasts, resulting in a net loss of bone over time. Several studies have demonstrated that intermittent administration of teriparatide has the surprising effect of increasing bone density (see Deal, Current Rheum. Reports 6:49-58, 2004). A brief exposure to high plasma levels of teriparatide seem to produce a greater benefit to osteoporosis patients than sustained high levels of this therapeutic. Accordingly, there is a demand to routes of administration of teriparatide that will provide higher plasma concentrations of the therapeutic for shorter periods of time, and which are easier for a patient to use that the current marketed subcutaneous formulation. Most recently, intermittent use of teriparatide has been recently been tested in combination therapy with alendronate, an antiresorptive, in osteoporosis patients (Black, et al., NEJM 353:555-565, 2005; Cosman, et al., ib., pp. 566-75). The beneficial prospects for combination therapy provide an increasing demand for additional modes of administration that permit patient control over teriparatide blood levels.

It would be preferable for patient acceptability if a non-injected route of administration were available, such as nasal, bucal, gastrointestinal or dermal. Teriparatide has been previously been administered intranasally to humans at doses of up to 500 μg per day for 7 days in one study (Suntory News Release. Suntory Establishes Large Scale Production of recombinant human PTH₁₋₃₄ and obtains promising results from Phase 1 Clinical Trials using a Nasal Formulation, February 1999 (http://www.suntory .com/news/1999-02.html, accessed 15 Apr. 2004) and in another study subjects received up to 1,000 μg per day for 3 months (Matsumoto, et al., “Daily Nasal Spray of hPTH₁₋₃₄ for 3 Months Increases Bone Mass In Osteoporotic Subjects,” ASBMR, presentation 1171, Seattle, Oct. 4, 2004). No safety concerns were noted with this route.

In general, many patients are adverse to injection of a drug and compliance with the prescribed dosing of PTH by injection is a problem. What is needed is an intranasal formulation of a PTH drug that is effective to treat osteoporosis or osteopenia.

### SUMMARY OF THE INVENTION

One aspect of the invention is a method for treating osteoporosis in a mammal, preferably a human patient, comprising administering intranasally a therapeutically effective amount of a PTH formulation to the mammal combined with administration of an anti-resorptive agent, in which the PTH formulation comprises teriparatide. In one embodiment, time to maximum plasma concentration, Tₘₐₓ, of said teriparatide following administration of said formulation to the mammal is less than 30 minutes, preferably about 20 minutes. In a related embodiment, elimination half life is about 55 minutes. In another embodiment, Cₘₐₓ is greater than about 75 pg/ml, preferably greater than about 150 pmole/l, more preferably greater than about 300 pg/ml. In another embodiment, AUC is greater than about 150 pg hr/ml.

Another aspect of the invention is a method for treating osteoporosis in a mammal, preferably a human patient, comprising administering intranasally a therapeutically effective amount of a PTH formulation to the mammal combined with administration of an anti-resorptive agent, in which the anti-resorptive agent is a bisphosphonate. In one embodiment, the bisphosphonate is selected from the group consisting of alendronate, ibandronate, risedronate, clodronate, etridonate, raloxifene, and mixtures thereof. In another embodiment, the anti-resorptive agent is a hormone, preferably an estrogen or a progesterone. In another embodiment, the anti-resorptive agent is a selective estrogen receptor modulator (SERM), preferably raloxifene.

Another aspect of the invention is a method for treating osteoporosis in a mammal comprising administering intranasally a therapeutically effective amount of a PTH formulation to the mammal combination with a bisphosphonate wherein the PTH formulation comprises a PTH peptide selected from the group consisting of SEQ NO: 1, SEQ NO: 2, and SEQ NO: 3, and wherein a time to maximum plasma concentration, Tₘₐₓ, of said PTH peptide following administration of said formulation to the mammal is less than about 30 minutes, preferably in which Cₘₐₓ is greater than about 150 pmole/l.

### BRIEF DESCRIPTION OF THE DRAWINGS

FIG. 1 shows the pharmacokinetics and plasma levels of teriparatide following administration of three formulations. Two IN formulations (5014, open circles; PTH-061, open diamonds) are shown along with a SC formulation (FORSTEO, open squares).

### DISCLOSURE OF THE INVENTION

Preferably the parathyroid hormone and the mammal are human. In a preferred embodiment the parathyroid hormone peptide, is PTH₁₋₃₄. Tregear, U.S. Pat. No. 4,086,196, described human PTH analogues and claimed that the first 27 to 34 amino acids are the most effective in terms of the stimulation of adenylyl cyclase in an in vitro cell assay. Pang, et al., WO 93/06845, published Apr. 15, 1993, described analogues of hPTH which involve substitutions of Arg²⁵, Lys²⁶, Lys²⁷ with numerous amino acids, including alanine, asparagine, aspartic acid, cysteine, glutamine, glutamic acid, glycine, histidine, isoleucine, leucine, methionine, phenylalanine, proline, serine, threonine, tryptophan, tyrosine, or valine. Other PTH analogues are disclosed in the following patents, hereby incorporated by reference: U.S. Pat. Nos. 5,317,010; 4,822,609; 5,693,616; 5,589,452; 4,833,125; 5,607,915; 5,556,940; 5,382,658; 5,407,911; 6,583,114; 6,541,450; 6,376,502; 5,955,425; 6,316,410; 6,110,892; 6,051,686; 5,695,955; 4,771,124; and 6,376,502.

PTH operates through activation of two second messenger systems, G_{S} -protein activated adenylyl cyclase (AC) and G_{q}-protein activated phospholipase C_{β}. The latter results in a stimulation of membrane-bound protein kinase CS (PKC) activity. The PKC activity has been shown to require PTH residues 29 to 32 (Jouishomme, et al., J. Bone Mineral Res. 9:1179-1189, 1994. It has been established that the increase in bone growth, i.e., that effect which is useful in the treatment of osteoporosis, is coupled to the ability of the peptide sequence to increase AC activity. The native PTH sequence has been shown to have all of these activities. The hPTH-(1-34) sequence is typically shown as:

(SEQ ID NO:2)

Ser Val Ser Glu Ile Gln Leu Met His Asn Leu Gly
Lys His Leu Asn Ser Met Glu Arg Val Glu Trp Leu
Arg Lys Lys Leu Gln Asp Val His Asn Phe.

The following linear analogue, hPTH₁₋₃₁NH₂, has only AC-stimulating activity and has been shown to be fully active in the restoration of bone loss in the ovariectomized rat model [Rixon, R. H., et al., J. Bone Miner. Res. 9:1179-1189, 1994; Whitfield, et al., Calcified Tissue Int. 58:81-87, 1996]; Willick, et al., U.S. Pat. No. 5,556,940, hereby incorporated by reference:

(SEQ ID NO:3)

Ser Val Ser Glu Ile Gln Leu Met His Asn Leu Gly
Lys His Leu Asn Ser Met Glu Arg Val Glu Trp Leu
Arg Lys Lys Leu Gln Asp Val.

The above molecule, SEQ ID NO:3, and its counterpart with a Leu₂₇ substitution SEQ ID NO:2 may have a free carboxyl ending instead of the amide ending. Another PTH analog is [Leu₂₇]cyclo(Glu₂₂-Lys₂₆)PTH₁₋₃₁.

This invention includes a method for treating osteoporosis or osteopenia in a mammal comprising transmucosally administering a formulation comprised of a PTH peptide, such that when at 50 μg of the PTH is administered transmucosally to the mammal the concentration of the PTH peptide in the plasma of the mammal increases by at least 5 pmol, preferably at least 10 pmol per liter of plasma.

Intranasal delivery-enhancing agents may be employed which enhance delivery of PTH into or across a nasal mucosal surface. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The intranasal delivery-enhancing agent of the present invention may be a pH control agent. The pH of the pharmaceutical formulation of the present invention is a factor affecting absorption of PTH via paracellular and transcellular pathways to drug transport. In one embodiment, the pharmaceutical formulation of the present invention is pH adjusted to between about pH 3.0 to 6.5. In a further embodiment, the pharmaceutical formulation of the present invention is pH adjusted to between about pH 3.0 to 5.0. In a further embodiment, the pharmaceutical formulation of the present invention is pH adjusted to between about pH 4.0 to 5.0. In other embodiments, the pH is 5.0±0.3.

As noted above, the present invention provides improved methods and compositions for mucosal delivery of PTH peptide to mammalian subjects for treatment or prevention of osteoporosis or osteopenia. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

According to the present invention a parathyroid hormone peptide also includes the free bases, acid addition salts or metal salts, such as potassium or sodium salts of the peptides, and parathyroid hormone peptides that have been modified by such processes as amidation, glycosylation, acylation, sulfation, phosphorylation, acetylation, cyclization and other well known covalent modification methods.

Osteopenia is a decreased calcification or density of bone, a descriptive term applicable to all skeletal systems in which the condition is noted. “Mucosal delivery enhancing agents” are defined as chemicals and other excipients that, when added to a formulation comprising water, salts and/or common buffers and PTH peptide (the control formulation) produce a formulation that produces a significant increase in transport of PTH peptide across a mucosa as measured by the maximum blood, serum, or cerebral spinal fluid concentration (Cₘₐₓ) or by the area under the curve, AUC, in a plot of concentration versus time. A mucosa includes the nasal, oral, intestinal, buccal, bronchopulmonary, vaginal, and rectal mucosal surfaces and in fact includes all mucus-secreting membranes lining all body cavities or passages that communicate with the exterior. Mucosal delivery enhancing agents are sometimes called carriers. “Non-infused administration” means any method of delivery that does not involve an injection directly into an artery or vein, a method which forces or drives (typically a fluid) into something and especially to introduce into a body part by means of a needle, syringe or other invasive method. Non-infused administration includes subcutaneous injection, intramuscular injection, intraparitoneal injection and the non-injection methods of delivery to a mucosa.

As noted above, the instant invention provides improved and useful methods and compositions for nasal mucosal delivery of a PTH peptide to prevent and treat osteoporosis or osteopenia in mammalian subjects. As used herein, prevention and treatment of osteoporosis or osteopenia means prevention of the onset or lowering the incidence or severity of clinical osteoporosis by reducing increasing bone mass, decreasing bone resorption or reducing the incidence of fractured bones in a patient.

The PTH peptide can also be administered in conjunction with other therapeutic agents such as bisphonates, calcium, vitamin D, estrogen or estrogen-receptor binding compounds, selective estrogen receptor modulators (SERMs), bone morphogenic proteins or calcitonin.

Improved methods and compositions for mucosal administration of PTH peptide to mammalian subjects utilize PTH peptide dosing schedules. The present invention provides mucosal delivery of PTH peptide formulated with one or more mucosal delivery-enhancing agents wherein PTH peptide dosage release is substantially normalized and/or sustained for an effective delivery period of PTH peptide release ranges from approximately 0.1 to 2.0 hours; 0.4 to 1.5 hours; 0.7 to 1.5 hours; or 0.8 to 1.0 hours; following mucosal administration. The sustained release of PTH peptide achieved may be facilitated by repeated administration of exogenous PTH peptide utilizing methods and compositions of the present invention.

Improved compositions and methods for mucosal administration of PTH peptide to mammalian subjects optimize PTH peptide dosing schedules. The present invention provides improved mucosal (e.g., nasal) delivery of a formulation comprising PTH peptide in combination with one or more mucosal delivery-enhancing agents and an optional sustained release-enhancing agent or agents. Mucosal delivery-enhancing agents of the present invention yield an effective increase in delivery, e.g., an increase in the maximal plasma concentration (Cₘₐₓ) to enhance the therapeutic activity of mucosally-administered PTH peptide. A second factor affecting therapeutic activity of PTH peptide in the blood plasma and CNS is residence time (RT). Sustained release-enhancing agents, in combination with intranasal delivery-enhancing agents, increase Cₘₐₓ and increase residence time (RT) of PTH peptide. Polymeric delivery vehicles and other agents and methods of the present invention that yield sustained release-enhancing formulations, for example, polyethylene glycol (PEG), are disclosed herein. The present invention provides an improved PTH peptide delivery method and dosage form for treatment or prevention of osteoporosis or osteopenia in mammalian subjects.

Within the mucosal delivery formulations and methods of the invention, the PTH peptide is frequently combined or coordinately administered with a suitable carrier or vehicle for mucosal delivery. As used herein, the term “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. Examples of ingredients of the above categories can be found in the U.S. Pharmacopeia National Formulary, 1990, pp. 1857-1859, as well as in Raymond C. Rowe, et al., Handbook of Pharmaceutical Excipients, 5th ed., 2006, and “Remington: The Science and Practice of Pharmacy,” 21st ed., 2006, editor David B. Troy.

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

Within the mucosal delivery compositions and methods of the invention, various delivery-enhancing agents are employed which enhance delivery of PTH peptide into or across a mucosal surface. In this regard, delivery of PTH peptide across the mucosal epithelium can occur “transcellularly” or “paracellularly.” The extent to which these pathways contribute to the overall flux and bioavailability of the PTH peptide depends upon the environment of the mucosa, the physico-chemical properties the active agent, and on the properties of the mucosal epithelium. Paracellular transport involves only passive diffusion, whereas transcellular transport can occur by passive, facilitated or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected PTH peptide within the invention. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against macromolecular permeation would suggest that the paracellular route would be a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively or within a single method or composition.

As used herein, “mucosal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of PTH peptide or other biologically active compound(s). Enhancement of mucosal delivery can thus occur by any of a variety of mechanisms, for example by increasing the diffusion, transport, persistence or stability of PTH peptide, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, a “mucosally effective amount of PTH peptide” contemplates effective mucosal delivery of PTH peptide to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of PTH peptide in a blood plasma”, “area under concentration vs. time curve (AUC) of PTH peptide in a blood plasma”, “time to maximal plasma concentration (tₘₐₓ) of PTH peptide in a blood plasma” are pharmacokinetic parameters known to one skilled in the art. Laursen, et al., Eur. J. Endocrinology 135:309-315, 1996. The “concentration vs. time curve” measures the concentration of PTH peptide in a blood serum of a subject vs. time after administration of a dosage of PTH peptide to the subject either by intranasal, intramuscular, subcutaneous, or other parenteral route of administration. “Cₘₐₓ” is the maximum concentration of PTH peptide in the blood serum of a subject following a single dosage of PTH peptide to the subject. “tₘₐₓ” is the time to reach maximum concentration of PTH peptide in a blood serum of a subject following administration of a single dosage of PTH peptide to the subject.

While the mechanism of absorption promotion may vary with different mucosal delivery-enhancing agents of the invention, useful reagents in this context will not substantially adversely affect the mucosal tissue and is selected according to the physicochemical characteristics of the particular PTH peptide or other active or delivery-enhancing agent. In this context, delivery-enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption-promoting agents for coordinate administration or combinatorial formulation with PTH peptide of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the PTH peptide. In additional aspects, surfactants (e.g., polysorbates) are employed as adjunct compounds, processing agents, or formulation additives to enhance intranasal delivery of the PTH peptide. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the PTH peptide from the vehicle into the mucosa.

Additional mucosal delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4--which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the PTH peptide. These include, inter alia, cyclodextrins and β-cyclodextrin derivatives (e.g., 2-hydroxypropyl-β-cyclodextrin and heptakis(2,6-di-O-methyl-β-cyclodextrin). These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate--extracts of coconut oil, Capmul), and triglycerides (e.g., amylodextrin, Estaram 299, Miglyol 810).

The mucosal therapeutic and prophylactic compositions of the present invention may be supplemented with any suitable penetration-promoting agent that facilitates absorption, diffusion, or penetration of PTH peptide across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g., monoaminocarboxlic acids such as glycine, alanine, phenylalanine, proline, hydroxyproline, etc.; hydroxyamino acids such as serine; acidic amino acids such as aspartic acid, glutamic acid, etc.; and basic amino acids such as lysine etc.—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g., sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved nasal mucosal delivery formulations and methods are provided that allow delivery of PTH peptide and other therapeutic agents within the invention across mucosal barriers between administration and selected target sites. Certain formulations are specifically adapted for a selected target cell, tissue or organ, or even a particular disease state. In other aspects, formulations and methods provide for efficient, selective endo- or transcytosis of PTH peptide specifically routed along a defined intracellular or intercellular pathway. Typically, the PTH peptide is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The PTH peptide may be provided in a delivery vehicle or otherwise modified (e.g., in the form of a prodrug), wherein release or activation of the PTH peptide is triggered by a physiological stimulus (e.g. pH change, lysosomal enzymes, etc.). Often, the PTH peptide is pharmacologically inactive until it reaches its target site for activity. In most cases, the PTH peptide and other formulation components are non-toxic and non-immunogenic. In this context, carriers and other formulation components are generally selected for their ability to be rapidly degraded and excreted under physiological conditions. At the same time, formulations are chemically and physically stable in dosage form for effective storage.

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. A wide variety of useful analogs and mimetics of PTH peptide are contemplated for use within the invention and can be produced and tested for biological activity according to known methods. Often, the peptides or proteins of PTH peptide or other biologically active peptides or proteins for use within the invention are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine. By aligning a peptide or protein analog optimally with a corresponding native peptide or protein, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide or protein.

An approach for stabilizing solid protein formulations of the invention is to increase the physical stability of purified, e.g., lyophilized, protein. This will inhibit aggregation via hydrophobic interactions as well as via covalent pathways that may increase as proteins unfold. Stabilizing formulations in this context often include polymer-based formulations, for example a biodegradable hydrogel formulation/delivery system. As noted above, the critical role of water in protein structure, function, and stability is well known. Typically, proteins are relatively stable in the solid state with bulk water removed. However, solid therapeutic protein formulations may become hydrated upon storage at elevated humidities or during delivery from a sustained release composition or device. The stability of proteins generally drops with increasing hydration. Water can also play a significant role in solid protein aggregation, for example, by increasing protein flexibility resulting in enhanced accessibility of reactive groups, by providing a mobile phase for reactants, and by serving as a reactant in several deleterious processes such as beta-elimination and hydrolysis.

Protein preparations containing between about 6% to 28% water are the most unstable. Below this level, the mobility of bound water and protein internal motions are low. Above this level, water mobility and protein motions approach those of full hydration. Up to a point, increased susceptibility toward solid-phase aggregation with increasing hydration has been observed in several systems. However, at higher water content, less aggregation is observed because of the dilution effect.

In accordance with these principles, an effective method for stabilizing peptides and proteins against solid-state aggregation for mucosal delivery is to control the water content in a solid formulation and maintain the water activity in the formulation at optimal levels. This level depends on the nature of the protein, but in general, proteins maintained below their “monolayer” water coverage will exhibit superior solid-state stability.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form using a solubilization agent. A range of components and additives are contemplated for use within these methods and formulations. Exemplary of these solubilization agents are cyclodextrins (CDs), which selectively bind hydrophobic side chains of polypeptides. These CDs have been found to bind to hydrophobic patches of proteins in a manner that significantly inhibits aggregation. This inhibition is selective with respect to both the CD and the protein involved. Such selective inhibition of protein aggregation provides additional advantages within the intranasal delivery methods and compositions of the invention. Additional agents for use in this context include CD dimers, trimers and tetramers with varying geometries controlled by the linkers that specifically block aggregation of peptides and protein. Yet solubilization agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention.

To improve the transport characteristics of biologically active agents (including PTH peptide, other active peptides and proteins, and macromolecular and small molecule drugs) for enhanced delivery across hydrophobic mucosal membrane barriers, the invention also provides techniques and reagents for charge modification of selected biologically active agents or delivery-enhancing agents described herein. In this regard, the relative permeabilities of macromolecules is generally be related to their partition coefficients. The degree of ionization of molecules, which is dependent on the pKₐ of the molecule and the pH at the mucosal membrane surface, also affects permeability of the molecules. Permeation and partitioning of biologically active agents, including PTH peptide and analogs of the invention, for mucosal delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

Consistent with these general teachings, mucosal delivery of charged macromolecular species, including PTH peptide and other biologically active peptides and proteins, within the methods and compositions of the invention is substantially improved when the active agent is delivered to the mucosal surface in a substantially un-ionized, or neutral, electrical charge state.

Certain PTH peptide and other biologically active peptide and protein components of mucosal formulations for use within the invention is charge modified to yield an increase in the positive charge density of the peptide or protein. These modifications extend also to cationization of peptide and protein conjugates, carriers and other delivery forms disclosed herein. Cationization offers a convenient means of altering the biodistribution and transport properties of proteins and macromolecules within the invention. Cationization is undertaken in a manner that substantially preserves the biological activity of the active agent and limits potentially adverse side effects, including tissue damage and toxicity.

Effective delivery of biotherapeutic agents via intranasal administration must take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant invention optionally incorporate effective mucolytic or mucus-clearing agents, which serve to degrade, thin or clear mucus from intranasal mucosal surfaces to facilitate absorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate >taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²+ and/or Mg²+ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited. In contrast, bacterial glycosidases which allow these microorganisms to permeate mucus layers of their hosts.

For combinatorial use with most biologically active agents within the invention, including peptide and protein therapeutics, non-ionogenic detergents are generally also useful as mucolytic or mucus-clearing agents. These agents typically will not modify or substantially impair the activity of therapeutic polypeptides.

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Ciliostatic agents find use within the methods and compositions of the invention to increase the residence time of mucosally (e.g., intranasally) administered PTH peptide, analogs and mimetics, and other biologically active agents disclosed herein. In particular, the delivery these agents within the methods and compositions of the invention may be significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of PTH peptide, analogs and mimetics, and other biologically active agents disclosed herein, without unacceptable adverse side effects.

Within more detailed aspects, a specific ciliostatic factor can be employed in a combined formulation or coordinate administration protocol with one or more PTH peptide proteins, analogs and mimetics, and/or other biologically active agents disclosed herein. Various bacterial ciliostatic factors isolated and characterized in the literature may be employed within these embodiments of the invention. Ciliostatic factors from the bacterium Pseudomonas aeruginosa include a phenazine derivative, a pyo compound (2-alkyl-4-hydroxyquinolines), and a rhamnolipid (also known as a hemolysin). The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis, which was associated with altered ciliary membranes. More extensive exposure to rhamnolipid was associated with removal of dynein arms from axonemes.

Within more detailed aspects of the invention, one or more membrane penetration-enhancing agents may be employed within a mucosal delivery method or formulation of the invention to enhance mucosal delivery of PTH peptide analogs and mimetics, and other biologically active agents disclosed herein. Membrane penetration enhancing agents in this context can be selected from: (i) a surfactant, (ii) a bile salt, (iii) a phospholipid additive, mixed micelle, liposome, or carrier, (iv) an alcohol, (v) an enamine, (vi) an NO donor compound, (vii) a long-chain amphipathic molecule (viii) a small hydrophobic penetration enhancer; (ix) sodium or a salicylic acid derivative; (x) a glycerol ester of acetoacetic acid (xi) a clyclodextrin or beta-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, (xviii) an inhibitor of cholesterol synthesis; or (xix) any combination of the membrane penetration enhancing agents recited in (i)-(xix).

Certain surface-active agents are readily incorporated within the mucosal delivery formulations and methods of the invention as mucosal absorption enhancing agents. These agents, which may be coordinately administered or combinatorially formulated with PTH peptide proteins, analogs and mimetics, and other biologically active agents disclosed herein, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF). The mechanisms of action of these various classes of surface-active agents typically include solubilization of the biologically active agent. For proteins and peptides which often form aggregates, the surface active properties of these absorption promoters can allow interactions with proteins such that smaller units such as surfactant coated monomers may be more readily maintained in solution. Examples of other surface-active agents are L-α-Phosphatidylcholine Didecanoyl (DDPC) polysorbate 80 and polysorbate 20. These monomers are presumably more transportable units than aggregates. A second potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

The present invention provides pharmaceutical composition that contains one or more PTH peptides, analogs or mimetics, and/or other biologically active agents in combination with mucosal delivery enhancing agents disclosed herein formulated in a pharmaceutical preparation for mucosal delivery.

The permeabilizing agent reversibly enhances mucosal epithelial paracellular transport, typically by modulating epithelial junctional structure and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occludins, or claudins. Examples of this are antibodies, antibody fragments or single-chain antibodies that bind to the extracellular domains of these proteins.

In yet additional detailed embodiments, the invention provides permeabilizing peptides and peptide analogs and mimetics for enhancing mucosal epithelial paracellular transport. The subject peptides and peptide analogs and mimetics typically work within the compositions and methods of the invention by modulating epithelial junctional structure and/or physiology in a mammalian subject. In certain embodiments, the peptides and peptide analogs and mimetics effectively inhibit homotypic and/or heterotypic binding of an epithelial membrane adhesive protein selected from a junctional adhesion molecule (JAM), occludin, or claudin.

One such agent that has been extensively studied is the bacterial toxin from Vibrio cholerae known as the “zonula occludens toxin” (ZOT). This toxin mediates increased intestinal mucosal permeability and causes disease symptoms including diarrhea in infected subjects. Fasano, et al., Proc. Nat. Acad. Sci., U.S.A. 8:5242-5246, 1991. When tested on rabbit ileal mucosa, ZOT increased the intestinal permeability by modulating the structure of intercellular tight junctions. More recently, it has been found that ZOT is capable of reversibly opening tight junctions in the intestinal mucosa. It has also been reported that ZOT is capable of reversibly opening tight junctions in the nasal mucosa. U.S. Pat. No. 5,908,825.

Within the methods and compositions of the invention, ZOT, as well as various analogs and mimetics of ZOT that function as agonists or antagonists of ZOT activity, are useful for enhancing intranasal delivery of biologically active agents—by increasing paracellular absorption into and across the nasal mucosa. In this context, ZOT typically acts by causing a structural reorganization of tight junctions marked by altered localization of the junctional protein ZO1. Within these aspects of the invention, ZOT is coordinately administered or combinatorially formulated with the biologically active agent in an effective amount to yield significantly enhanced absorption of the active agent, by reversibly increasing nasal mucosal permeability without substantial adverse side effects.

The compositions and delivery methods of the invention optionally incorporate a selective transport-enhancing agent that facilitates transport of one or more biologically active agents. These transport-enhancing agents may be employed in a combinatorial formulation or coordinate administration protocol with one or more of the PTH peptides, analogs and mimetics disclosed herein, to coordinately enhance delivery of one or more additional biologically active agent(s) across mucosal transport barriers, to enhance mucosal delivery of the active agent(s) to reach a target tissue or compartment in the subject (e.g., the mucosal epithelium, liver, CNS tissue or fluid, or blood plasma). Alternatively, the transport-enhancing agents may be employed in a combinatorial formulation or coordinate administration protocol to directly enhance mucosal delivery of one or more of the PTH peptides, analogs and mimetics, with or without enhanced delivery of an additional biologically active agent.

Exemplary selective transport-enhancing agents for use within this aspect of the invention include, but are not limited to, glycosides, sugar-containing molecules, and binding agents such as lectin binding agents, which are known to interact specifically with epithelial transport barrier components. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the invention. Certain bioadhesive ligands for use within the invention will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more PTH peptides, analogs and mimetics, and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within the invention. Lectins are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (e.g., concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

In addition to plant lectins, microbial adhesion and invasion factors provide a rich source of candidates for use as adhesive/selective transport carriers within the mucosal delivery methods and compositions of the invention. Two components are necessary for bacterial adherence processes, a bacterial ‘adhesin’ (adherence or colonization factor) and a receptor on the host cell surface. Bacteria causing mucosal infections need to penetrate the mucus layer before attaching themselves to the epithelial surface. This attachment is usually mediated by bacterial fimbriae or pilus structures, although other cell surface components may also take part in the process. Adherent bacteria colonize mucosal epithelia by multiplication and initiation of a series of biochemical reactions inside the target cell through signal transduction mechanisms (with or without the help of toxins). Associated with these invasive mechanisms, a wide diversity of bioadhesive proteins (e.g., invasin, intemalin) originally produced by various bacteria and viruses are known. These allow for extracellular attachment of such microorganisms with an impressive selectivity for host species and even particular target tissues. Signals transmitted by such receptor-ligand interactions trigger the transport of intact, living microorganisms into, and eventually through, epithelial cells by endo- and transcytotic processes. Such naturally occurring phenomena may be harnessed (e.g., by complexing biologically active agents such as PTH peptides with an adhesin) according to the teachings herein for enhanced delivery of biologically active compounds into or across mucosal epithelia and/or to other designated target sites of drug action.

Various bacterial and plant toxins that bind epithelial surfaces in a specific, lectin-like manner are also useful within the methods and compositions of the invention. For example, diptheria toxin (DT) enters host cells rapidly by RME. Likewise, the B subunit of the E. coli heat labile toxin binds to the brush border of intestinal epithelial cells in a highly specific, lectin-like manner. Uptake of this toxin and transcytosis to the basolateral side of the enterocytes has been reported in vivo and in vitro. Other researches have expressed the transmembrane domain of diphtheria toxin in E. coli as a maltose-binding fusion protein and coupled it chemically to high-Mw poly-L-lysine. The resulting complex was successfully used to mediate internalization of a reporter gene in vitro. In addition to these examples, Staphylococcus aureus produces a set of proteins (e.g., staphylococcal enterotoxin A (SEA), SEB, toxic shock syndrome toxin 1 (TSST- 1) which act both as superantigens and toxins. Studies relating to these proteins have reported dose-dependent, facilitated transcytosis of SEB and TSST- I in Caco-2 cells.

Viral haemagglutinins comprise another type of transport agent to facilitate mucosal delivery of biologically active agents within the methods and compositions of the invention. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus. These and other exemplary viral hemagglutinins can be employed in a combinatorial formulation (e.g., a mixture or conjugate formulation) or coordinate administration protocol with one or more of the PTH peptide, analogs and mimetics disclosed herein, to coordinately enhance mucosal delivery of one or more additional biologically active agent(s). Alternatively, viral hemagglutinins can be employed in a combinatorial formulation or coordinate administration protocol to directly enhance mucosal delivery of one or more of the PTH peptide proteins, analogs and mimetics, with or without enhanced delivery of an additional biologically active agent.

A variety of endogenous, selective transport-mediating factors are also available for use within the invention. Mammalian cells have developed an assortment of mechanisms to facilitate the internalization of specific substrates and target these to defined compartments. Collectively, these processes of membrane deformations are termed ‘endocytosis’ and comprise phagocytosis, pinocytosis, receptor-mediated endocytosis (clathrin-mediated RME), and potocytosis (non-clathrin-mediated RME). RME is a highly specific cellular biologic process by which, as its name implies, various ligands bind to cell surface receptors and are subsequently internalized and trafficked within the cell. In many cells the process of endocytosis is so active that the entire membrane surface is internalized and replaced in less than a half hour. Two classes of receptors are proposed based on their orientation in the cell membrane; the amino terminus of Type I receptors is located on the extracellular side of the membrane, whereas Type II receptors have this same protein tail in the intracellular milieu.

Still other embodiments of the invention utilize transferrin as a carrier or stimulant of RME of mucosally delivered biologically active agents. Transferrin, an 80 kDa iron-transporting glycoprotein, is efficiently taken up into cells by RME. Transferrin receptors are found on the surface of most proliferating cells, in elevated numbers on erythroblasts and on many kinds of tumors. The transcytosis of transferrin (Tf) and transferrin conjugates is reportedly enhanced in the presence of Brefeldin A (BFA), a fungal metabolite. In other studies, BFA treatment has been reported to rapidly increase apical endocytosis of both ricin and HRP in MDCK cells. Thus, BFA and other agents that stimulate receptor-mediated transport can be employed within the methods of the invention as combinatorially formulated (e.g., conjugated) and/or coordinately administered agents to enhance receptor-mediated transport of biologically active agents, including PTH peptide proteins, analogs and mimetics.

In certain aspects of the invention, the combinatorial formulations and/or coordinate administration methods herein incorporate an effective amount of peptides and proteins which may adhere to charged glass thereby reducing the effective concentration in the container. Silanized containers, for example, silanized glass containers, are used to store the finished product to reduce adsorption of the polypeptide or protein to a glass container.

In yet additional aspects of the invention, a kit for treatment of a mammalian subject comprises a stable pharmaceutical composition of one or more PTH peptide compound(s) formulated for mucosal delivery to the mammalian subject wherein the composition is effective for treating or preventing osteoporosis or osteopenia. The kit further comprises a pharmaceutical reagent vial to contain the one or more PTH peptide compounds. The pharmaceutical reagent vial is composed of pharmaceutical grade polymer, glass or other suitable material. The pharmaceutical reagent vial is, for example, a silanized glass vial. The kit further comprises an aperture for delivery of the composition to a nasal mucosal surface of the subject. The delivery aperture is composed of a pharmaceutical grade polymer, glass or other suitable material. The delivery aperture is, for example, a silanized glass.

A silanization technique combines a special cleaning technique for the surfaces to be silanized with a silanization process at low pressure. The silane is in the gas phase and at an enhanced temperature of the surfaces to be silanized. The method provides reproducible surfaces with stable, homogeneous and functional silane layers having characteristics of a monolayer. The silanized surfaces prevent binding to the glass of polypeptides or mucosal delivery enhancing agents of the present invention.

The procedure is useful to prepare silanized pharmaceutical reagent vials to hold PTH peptide compositions of the present invention. Glass trays are cleaned by rinsing with double distilled water (ddH₂O) before using. The silane tray is then be rinsed with 95% EtOH, and the acetone tray is rinsed with acetone. Pharmaceutical reagent vials are sonicated in acetone for 10 minutes. After the acetone sonication, reagent vials are washed in ddH₂O tray at least twice. Reagent vials are sonicated in 0.1M NaOH for 10 minutes. While the reagent vials are sonicating in NaOH, the silane solution is made under a hood. (Silane solution: 800 mL of 95% ethanol; 96 L of glacial acetic acid; 25 mL of glycidoxypropyltrimethoxy silane). After the NaOH sonication, reagent vials are washed in ddH₂O tray at least twice. The reagent vials are sonicated in silane solution for 3 to 5 minutes. The reagent vials are washed in 100% EtOH tray. The reagent vials are dried with prepurified N₂ gas and stored in a 100° C. oven for at least 2 hours before using.

In certain aspects of the invention, the combinatorial formulations and/or coordinate administration methods herein incorporate an effective amount of a nontoxic bioadhesive as an adjunct compound or carrier to enhance mucosal delivery of one or more biologically active agent(s). Bioadhesive agents in this context exhibit general or specific adhesion to one or more components or surfaces of the targeted mucosa. The bioadhesive maintains a desired concentration gradient of the biologically active agent into or across the mucosa to ensure penetration of even large molecules (e.g., peptides and proteins) into or through the mucosal epithelium. Typically, employment of a bioadhesive within the methods and compositions of the invention yields a two- to five- fold, often a five- to ten-fold increase in permeability for peptides and proteins into or through the mucosal epithelium. This enhancement of epithelial permeation often permits effective transmucosal delivery of large macromolecules, for example to the basal portion of the nasal epithelium or into the adjacent extracellular compartments or a blood plasma or CNS tissue or fluid.

This enhanced delivery provides for greatly improved effectiveness of delivery of bioactive peptides, proteins and other macromolecular therapeutic species. These results will depend in part on the hydrophilicity of the compound, whereby greater penetration is achieved with hydrophilic species compared to water insoluble compounds. In addition to these effects, employment of bioadhesives to enhance drug persistence at the mucosal surface can elicit a reservoir mechanism for protracted drug delivery, whereby compounds not only penetrate across the mucosal tissue but also back-diffuse toward the mucosal surface once the material at the surface is depleted.

A variety of suitable bioadhesives are disclosed in the art for oral administration, U.S. Pat. Nos. 3,972,995; 4,259,314; 4,680,323; 4,740,365; 4,573,996; 4,292,299; 4,715,369; 4,876,092; 4,855,142; 4,250,163; 4,226,848; 4,948,580; U.S. Pat. Reissue No. 33,093, hereby incorporated by reference, which find use within the novel methods and compositions of the invention. The potential of various bioadhesive polymers as a mucosal, e.g., nasal, delivery platform within the methods and compositions of the invention can be readily assessed by determining their ability to retain and release PTH peptide, as well as by their capacity to interact with the mucosal surfaces following incorporation of the active agent therein. In addition, well known methods is applied to determine the biocompatibility of selected polymers with the tissue at the site of mucosal administration. When the target mucosa is covered by mucus (i.e., in the absence of mucolytic or mucus-clearing treatment), it can serve as a connecting link to the underlying mucosal epithelium. Therefore, the term “bioadhesive” as used herein also covers mucoadhesive compounds useful for enhancing mucosal delivery of biologically active agents within the invention. However, adhesive contact to mucosal tissue mediated through adhesion to a mucus gel layer may be limited by incomplete or transient attachment between the mucus layer and the underlying tissue, particularly at nasal surfaces where rapid mucus clearance occurs. In this regard, mucin glycoproteins are continuously secreted and, immediately after their release from cells or glands, form a viscoelastic gel. The luminal surface of the adherent gel layer, however, is continuously eroded by mechanical, enzymatic and/or ciliary action. Where such activities are more prominent or where longer adhesion times are desired, the coordinate administration methods and combinatorial formulation methods of the invention may further incorporate mucolytic and/or ciliostatic methods or agents as disclosed herein above.

Typically, mucoadhesive polymers for use within the invention are natural or synthetic macromolecules which adhere to wet mucosal tissue surfaces by complex, but non-specific, mechanisms. In addition to these mucoadhesive polymers, the invention also provides methods and compositions incorporating bioadhesives that adhere directly to a cell surface, rather than to mucus, by means of specific, including receptor-mediated, interactions. One example of bioadhesives that function in this specific manner is the group of compounds known as lectins. These are glycoproteins with an ability to specifically recognize and bind to sugar molecules, e.g., glycoproteins or glycolipids, which form part of intranasal epithelial cell membranes and can be considered as “lectin receptors.”

In certain aspects of the invention, bioadhesive materials for enhancing intranasal delivery of biologically active agents comprise a matrix of a hydrophilic, e.g., water soluble or swellable, polymer or a mixture of polymers that can adhere to a wet mucous surface. These adhesives may be formulated as ointments, hydrogels (see above) thin films, and other application forms. Often, these adhesives have the biologically active agent mixed therewith to effectuate slow release or local delivery of the active agent. Some are formulated with additional ingredients to facilitate penetration of the active agent through the nasal mucosa, e.g., into the circulatory system of the individual.

Various polymers, both natural and synthetic ones, show significant binding to mucus and/or mucosal epithelial surfaces under physiological conditions. The strength of this interaction can readily be measured by mechanical peel or shear tests. When applied to a humid mucosal surface, many dry materials will spontaneously adhere, at least slightly. After such an initial contact, some hydrophilic materials start to attract water by adsorption, swelling or capillary forces, and if this water is absorbed from the underlying substrate or from the polymer-tissue interface, the adhesion may be sufficient to achieve the goal of enhancing mucosal absorption of biologically active agents. Such ‘adhesion by hydration’ can be quite strong, but formulations adapted to employ this mechanism must account for swelling which continues as the dosage transforms into a hydrated mucilage. This is projected for many hydrocolloids useful within the invention, especially some cellulose-derivatives, which are generally non-adhesive when applied in pre-hydrated state. Nevertheless, bioadhesive drug delivery systems for mucosal administration are effective within the invention when such materials are applied in the form of a dry polymeric powder, microsphere, or film-type delivery form.

Other polymers adhere to mucosal surfaces not only when applied in dry, but also in fully hydrated state, and in the presence of excess amounts of water. The selection of a mucoadhesive thus requires due consideration of the conditions, physiological as well as physico-chemical, under which the contact to the tissue is formed and maintained. In particular, the amount of water or humidity usually present at the intended site of adhesion, and the prevailing pH, are known to largely affect the mucoadhesive binding strength of different polymers.

Several polymeric bioadhesive drug delivery systems have been fabricated and studied in the past 20 years, not always with success. A variety of such carriers are, however, currently used in clinical applications involving dental, orthopedic, ophthalmological, and surgical uses. For example, acrylic-based hydrogels have been used extensively for bioadhesive devices. Acrylic-based hydrogels are well suited for bioadhesion due to their flexibility and nonabrasive characteristics in the partially swollen state, which reduce damage-causing attrition to the tissues in contact. Furthermore, their high permeability in the swollen state allows unreacted monomer, un-crosslinked polymer chains, and the initiator to be washed out of the matrix after polymerization, which is an important feature for selection of bioadhesive materials for use within the invention. Acrylic-based polymer devices exhibit very high adhesive bond strength. For controlled mucosal delivery of peptide and protein drugs, the methods and compositions of the invention optionally include the use of carriers, e.g., polymeric delivery vehicles that function in part to shield the biologically active agent from proteolytic breakdown, while at the same time providing for enhanced penetration of the peptide or protein into or through the nasal mucosa. In this context, bioadhesive polymers have demonstrated considerable potential for enhancing oral drug delivery. As an example, the bioavailability of 9-desglycinamide, 8-arginine vasopressin (DGAVP) intraduodenally administered to rats together with a 1% (w/v) saline dispersion of the mucoadhesive poly(acrylic acid) derivative polycarbophil, was 3-5-fold increased compared to an aqueous solution of the peptide drug without this polymer.

Mucoadhesive polymers of the poly(acrylic acid)-type are potent inhibitors of some intestinal proteases. The mechanism of enzyme inhibition is explained by the strong affinity of this class of polymers for divalent cations, such as calcium or zinc, which are essential cofactors of metallo-proteinases, such as trypsin and chymotrypsin. Depriving the proteases of their cofactors by poly(acrylic acid) was reported to induce irreversible structural changes of the enzyme proteins which were accompanied by a loss of enzyme activity. At the same time, other mucoadhesive polymers (e.g., some cellulose derivatives and chitosan) may not inhibit proteolytic enzymes under certain conditions. In contrast to other enzyme inhibitors contemplated for use within the invention (e.g., aprotinin, bestatin), which are relatively small molecules, the trans-nasal absorption of inhibitory polymers is likely to be minimal in light of the size of these molecules, and thereby eliminate possible adverse side effects. Thus, mucoadhesive polymers, particularly of the poly(acrylic acid)-type, may serve both as an absorption-promoting adhesive and enzyme-protective agent to enhance controlled delivery of peptide and protein drugs, especially when safety concerns are considered.

In addition to protecting against enzymatic degradation, bioadhesives and other polymeric or non-polymeric absorption-promoting agents for use within the invention may directly increase mucosal permeability to biologically active agents. To facilitate the transport of large and hydrophilic molecules, such as peptides and proteins, across the nasal epithelial barrier, mucoadhesive polymers and other agents have been postulated to yield enhanced permeation effects beyond what is accounted for by prolonged premucosal residence time of the delivery system. The time course of drug plasma concentrations reportedly suggested that the bioadhesive microspheres caused an acute, but transient increase of insulin permeability across the nasal mucosa. Other mucoadhesive polymers for use within the invention, for example chitosan, reportedly enhance the permeability of certain mucosal epithelia even when they are applied as an aqueous solution or gel. Another mucoadhesive polymer reported to directly affect epithelial permeability is hyaluronic acid and ester derivatives thereof A particularly useful bioadhesive agent within the coordinate administration, and/or combinatorial formulation methods and compositions of the invention is chitosan, as well as its analogs and derivatives. Chitosan is a non-toxic, biocompatible and biodegradable polymer that is widely used for pharmaceutical and medical applications because of its favorable properties of low toxicity and good biocompatibility. It is a natural polyaminosaccharide prepared from chitin by N-deacetylation with alkali. As used within the methods and compositions of the invention, chitosan increases the retention of PTH peptides, analogs and mimetics, and other biologically active agents disclosed herein at a mucosal site of application. This mode of administration can also improve patient compliance and acceptance. As further provided herein, the methods and compositions of the invention will optionally include a novel chitosan derivative or chemically modified form of chitosan. One such novel derivative for use within the invention is denoted as a β-[1→4]-2-guanidino-2-deoxy-D-glucose polymer (poly-GuD). Chitosan is the N-deacetylated product of chitin, a naturally occurring polymer that has been used extensively to prepare microspheres for oral and intra-nasal formulations. The chitosan polymer has also been proposed as a soluble carrier for parenteral drug delivery. Within one aspect of the invention, o-methylisourea is used to convert a chitosan amine to its guanidinium moiety. The guanidinium compound is prepared, for example, by the reaction between equi-normal solutions of chitosan and o-methylisourea at pH above 8.0.

The guanidinium product is -[14]-guanidino-2-deoxy-D-glucose polymer. It is abbreviated as Poly-GuD in this context (Monomer F.W. of Amine in Chitosan=161; Monomer F.W. of Guanidinium in Poly-GuD=203).

Additional compounds classified as bioadhesive agents for use within the present invention act by mediating specific interactions, typically classified as “receptor-ligand interactions” between complementary structures of the bioadhesive compound and a component of the mucosal epithelial surface. Many natural examples illustrate this form of specific binding bioadhesion, as exemplified by lectin-sugar interactions. Lectins are (glyco) proteins of non-immune origin which bind to polysaccharides or glycoconjugates.

Several plant lectins have been investigated as possible pharmaceutical absorption-promoting agents. One plant lectin, Phaseolus vulgaris hemagglutinin (PHA), exhibits high oral bioavailability of more than 10% after feeding to rats. Tomato (Lycopersicon esculeutum) lectin (TL) appears safe for various modes of administration.

In summary, the foregoing bioadhesive agents are useful in the combinatorial formulations and coordinate administration methods of the instant invention, which optionally incorporate an effective amount and form of a bioadhesive agent to prolong persistence or otherwise increase mucosal absorption of one or more PTH peptides, analogs and mimetics, and other biologically active agents. The bioadhesive agents may be coordinately administered as adjunct compounds or as additives within the combinatorial formulations of the invention. In certain embodiments, the bioadhesive agent acts as a ‘pharmaceutical glue’, whereas in other embodiments adjunct delivery or combinatorial formulation of the bioadhesive agent serves to intensify contact of the biologically active agent with the nasal mucosa, in some cases by promoting specific receptor-ligand interactions with epithelial cell “receptors,” and in others by increasing epithelial permeability to significantly increase the drug concentration gradient measured at a target site of delivery (e.g., liver, blood plasma, or CNS tissue or fluid). Yet additional bioadhesive agents for use within the invention act as enzyme (e.g., protease) inhibitors to enhance the stability of mucosally administered biotherapeutic agents delivered coordinately or in a combinatorial formulation with the bioadhesive agent.

The coordinate administration methods and combinatorial formulations of the instant invention optionally incorporate effective lipid or fatty acid based carriers, processing agents, or delivery vehicles, to provide improved formulations for mucosal delivery of PTH peptides, analogs and mimetics, and other biologically active agents. For example, a variety of formulations and methods are provided for mucosal delivery which comprise one or more of these active agents, such as a peptide or protein, admixed or encapsulated by, or coordinately administered with, a liposome, mixed micellar carrier, or emulsion, to enhance chemical and physical stability and increase the half life of the biologically active agents (e.g., by reducing susceptibility to proteolysis, chemical modification and/or denaturation) upon mucosal delivery.

Additional methods and compositions provided within the invention involve chemical modification of biologically active peptides and proteins by covalent attachment of polymeric materials, for example dextrans, polyvinyl pyrrolidones, glycopeptides, polyethylene glycol and polyamino acids. The resulting conjugated peptides and proteins retain their biological activities and solubility for mucosal administration. In alternate embodiments, PTH peptide proteins, analogs and mimetics, and other biologically active peptides and proteins, are conjugated to polyalkylene oxide polymers, particularly polyethylene glycols (PEG). U.S. Pat. No. 4,179,337, hereby incorporated by reference.

Peptides could be linked to PEG directly as described in the art. PEG can be a molecule having a molecular mass ranging between 300 and 60,000. Also included are various PEG molecules, including linear, branched, attached to a peptide at a single moiety or multiple attachment sites. Amine-reactive PEG polymers for use within the invention include SC-PEG with molecular masses of 2000, 5000, 10000, 12000, and 20 000; U-PEG-10000; NHS-PEG-3400-biotin; T-PEG-5000; T-PEG-12000; and TPC-PEG-5000. PEGylation of biologically active peptides and proteins may be achieved by modification of carboxyl sites (e.g., aspartic acid or glutamic acid groups in addition to the carboxyl terminus). The utility of PEG-hydrazide in selective modification of carbodiimide-activated protein carboxyl groups under acidic conditions has been described. Alternatively, bifunctional PEG modification of biologically active peptides and proteins can be employed. In some procedures, charged amino acid residues, including lysine, aspartic acid, and glutamic acid, have a marked tendency to be solvent accessible on protein surfaces.

In addition to PEGylation, biologically active agents such as peptides and proteins for use within the invention can be modified to enhance circulating half-life by shielding the active agent via conjugation to other known protecting or stabilizing compounds, for example by the creation of fusion proteins with an active peptide, protein, analog or mimetic linked to one or more carrier proteins, such as one or more immunoglobulin chains.

Mucosal delivery formulations of the present invention comprise PTH peptides, analogs and mimetics, typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069, hereby incorporated by reference. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 5.0±0.3. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

A nasal spray device can be selected according to what is customary in the industry or acceptable by the regulatory health authorities. One example of a suitable device is described in described in U.S. application Ser. No. 10/869,649 (S. Quay and G. Brandt: Compositions and methods for enhanced mucosal delivery of Y2 receptor-binding peptides and methods for treating and preventing obesity, filed Jun. 16, 2004).

To treat osteoporosis or osteopenia, an intranasal dose of a PTH peptide parathyroid hormone is administered at dose high enough to promote an increase in bone mass but low enough so as not to induce any unwanted side-effects such as nausea. A preferred intranasal dose of a PTH peptide such as parathyroid hormone(1-34) is about 3 μg -10 μg/kg weight of the patient, most preferably about 6 μg/kg weight of the patient. In a standard dose a patient will receive 50 μg to 800 μg, more preferably about between 100 μg to 400 μg, most preferably 150 μg to about 200 μg. The PTH peptide such as parathyroid hormone (1-34) is preferably administered once a day.

All publications, references, patents, patent publications and patent applications cited herein are each hereby specifically incorporated by reference in their entirety.

While this invention has been described in relation to certain embodiments, and many details have been set forth for purposes of illustration, it will be apparent to those skilled in the art that this invention includes additional embodiments, and that some of the details described herein may be varied considerably without departing from this invention. This invention includes such additional embodiments, modifications and equivalents. In particular, this invention includes any combination of the features, terms, or elements of the various illustrative components and examples.

The use herein of the terms “a,” “an,” “the,” and similar terms in describing the invention, and in the claims, are to be construed to include both the singular and the plural. The terms “comprising,” “having,” “including,” and “containing” are to be construed as open-ended terms which mean, for example, “including, but not limited to.” Recitation of a range of values herein refers individually to each separate value falling within the range as if it were individually recited herein, whether or not some of the values within the range are expressly recited. Specific values employed herein will be understood as exemplary and non to limit the scope of the invention.

The examples given herein, and the exemplary language used herein are solely for the purpose of illustration, and are not intended to limit the scope of the invention.

### EXAMPLE

### Example 1

### Stability

Teriparatide Nasal Spray will be supplied to the clinic as a liquid in a vial for 5 intranasal administration via an actuator. Details for formulation compositions between 1.0 and 4.0 mg/mL Teriparatide strengths are shown below.

| | |
|---|---|
| | Unit Strength* |
| Components | mg/mL |

| | | |
|---|---|---|
| Teriparatide | 1 | 1.5 |
| Chlorobutanol | 5 | 5 |
| Methyl-β-Cyclodextrin | 45 | 45 |
| L-Alpha-Phosphatidylcholine Didecanoyl | 1 | 1 |
| Edetate Disodium | 1 | 1 |
| Sorbitol | 26 | 26 |
| Hydrochloric Acid^{†} | — | — |
| Sodium Hydroxide^{†} | — | — |
| Purified Water, USP or Sterile Water | q.s. | q.s. |
| for Irrigation | | |
| Teriparatide | 3 | 4 |
| Chlorobutanol | 5 | 5 |
| Polysorbate 80 | 1 | 1 |
| Sorbitol | 31 | 31 |
| Hydrochloric Acid^{†} | — | — |
| Sodium Hydroxide^{†} | — | — |
| Purified Water, USP or Sterile Water | q.s. to | q.s. to |
| for Irrigation | 1.0 mL | 1.0 mL |

This solution is provided in a multi-unit dose container to deliver a metered dose of 0.1 mL of drug product per actuation. Hydrochloric acid is added for pH adjustment to meet target pH of 4.0±0.2. The stability of the formulations were monitored at regular intervals. The results show teriparatide nasal sprays of the invention may be safely stored at 5° C. and 25° C. for four weeks without sterilization. Comparable results were obtained with sodium benzoate substituted as a preservative.

### Example 2

### Pharmacokinetics in Human Subiects

The absorption and safety of two formulations of teriparatide nasal spray of the invention were evaluated at two dose levels. The bioavailability of FORSTEO (Eli Lilly UK) given subcutaneously was compared with that of two formulations of teriparatide nasal spray of the invention at two dose levels.

This study was a single-site, open-label, active controlled, 5 period crossover, dose ranging study involving 6 healthy male and 6 healthy female volunteers. The commercially available formulation of teriparatide, FORSTEO was the active control. The five study periods were as follows:

Period 1: All subjects received FORSTEO 20 μg subcutaneously.

Period 2: All subjects received 500 μg intranasal dose of test formulation.

Period 3: All subjects received 200 μg intranasal dose of test formulation

Period 4: All subjects received a 1000 μg intranasal dose of test formulation.

Period 5: All subjects received a 400 μg intranasal dose of test formulation

Blood samples for PK were collected at 0 (i.e., pre-dose), 5, 10, 15, 30, 45, 60, 90 minutes and 2, 3, and 4 hours post-dose and analyzed using a validated method. Because the bioassay is fully cross reactive with endogenous PTH(1-84), all data was corrected for pre-dose values. When this correction resulted in a negative post-dose value, all such negative values were set to ‘missing.’ Values reported as <LLOQ were set to half LLOQ in order to evaluate PK and change from baseline. Standard pharmacokinetic parameters, including AUCₗₐₛₜ, AUC_{inf}, Cₘₐₓ, t_{1/2}, tₘₐₓ, and Kₑ were calculated using WinNonlin. Intra-subject variability of the pharmacokinetic profiles was evaluated for the test versus the reference using analysis of variance methods. An analysis of variance (ANOVA) was performed based on a 2-period design and incorporating a main effect term for each of the two products under consideration (Snedecor, G. W. and W. G. Cochran, Statistical Methods, 6th ed., “One-Way Classifications—Analysis of Variance,” Iowa State University Press, Ames, IA, 1967, pp. 258-98). (Subject (Sequence) was a random effect in the model with all others fixed.) A separate model was created for each dose of teriparatide nasal spray versus the reference. The 90% confidence intervals were generated for the ratio of test dose/reference with respect to Cₘₐₓ, AUCₗₐₛₜ, and AUC_{inf}. These values were natural log (ln)-transformed prior to analysis. The corresponding 90% confidence intervals for the geometric mean ratio were obtained by taking the antilog of the 90% confidence intervals for the difference between the means on the log scale. These analyses were not performed to demonstrate bioequivalence but were for informational purposes only. As a result, no adjustment to the confidence level for each of the paired comparisons was made to account for multiplicity of analysis. This study is hypothesis-generating only. For tₘₐₓ, the analyses were run using Wilcoxon's signed-rank test (Steinijans, V. W. and E. Diletti, Eur. J. Clin. Pharmacol. 24:127-36, 1983, to determine if differences existed between a given test group and the reference group.

For each subject, the following PK parameters were calculated, whenever possible, based on the plasma concentrations of teriparatide for each test article, according to the model independent approach:

Cₘₐₓ Maximum observed concentration.

tₘₐₓ Time to maximum concentration.

AUCₗₐₛₜ Area under the concentration-time curve from time 0 to the time of last measurable concentration, calculated by the linear trapezoidal rule.

The following parameters were calculated when the data permitted accurate estimation of these parameters:

AUC_{inf} Area under the concentration-time curve extrapolated to infinity, calculated using the formula:

AUC_{inf}=AUCₗₐₛₜ+Cₜ/Kₑ where Cₜ is the last measurable concentration and Kₑ is the apparent terminal phase rate constant.

Kₑ Apparent terminal phase rate constant, where Kₑ is the magnitude of the slope of the linear regression of the log concentration versus time profile during the terminal phase.

t_{1/2} Apparent terminal phase half-life (whenever possible), where t_{1/2}=(ln2)/Kₑ.

All data was corrected for pre-dose values. When this correction resulted in a negative post-dose value, all such negative values were set to ‘missing.’ Values reported as <LLOQ were set to half LLOQ in order to evaluate pK and change from baseline. Actual (not nominal) sampling times were used in the calculation of all PK parameters.

A summary of arithmetic mean pharmacokinetic parameters for each formulation and dose of teriparatide are presented in Table 1. The mean tₘₐₓ was 0.68 versus 0.57 and 0.17 hours for the FORSTEO and low dose nasal formulations of PTH-061 and 05014, respectively. The Cₘₐₓ was 1.6 and 2.4 fold higher then FORSTEO for each low dose formulation. The AUCₗₐₛₜ was 1.23 and 1.45 fold higher then FORSTEO for each low dose formulation.

TABLE 1
Arithmetic Mean Pharmacokinetic Parameters by Formulation and Dose

| | Dose | Tmax | Cmax | AUClast | AUCinf | t½ | Ke |
|---|---|---|---|---|---|---|---|
| Formulation | (μg) | (hr) | (pg/mL) | (hr*pg/mL) | (hr*pg/mL) | (hr) | (1/hr) |
| FORSTEO | 20 | 0.68 | 70.80 | 85.92 | 132.12 | 1.57 | 0.638 |
| PTH-061 | 500 | 0.57 | 112.72 | 106.08 | 195.69 | 1.38 | 0.610 |
| PTH-061 | 1000 | 0.46 | 405.57 | 335.20 | 412.47 | 1.03 | 0.782 |
| 05014 | 200 | 0.17 | 172.72 | 125.07 | 269.60 | 3.10 | 0.720 |
| 05014 | 400 | 0.18 | 349.62 | 206.02 | 238.26 | 1.12 | 1.097 |

In addition, the tₘₐₓ results for each formulation were compared to the FORSTEO control using a simple Wilcoxon signed-rank test. The results (as p-values) are given in

TABLE 2
Comparison of Tₘₐₓ - FORSTEO and Nasal Formulations

| | p-value from Wilcoxon Signed- |
|---|---|
| Comparison of Tₘₐₓ | Rank Test |
| FORSTEO vs. Formulation, 500 μg | P = 0.75 |
| FORSTEO vs. Formulation, 1000 μg | P = 0.53 |
| FORSTEO vs. Formulation, 200 μg | P = 0.10 |
| FORSTEO vs. Formulation, 400 μg | P = 0.24 |

Thus, there does not appear to be differences in the tₘₐₓ values among the formulations with respect to FORSTEO.

The 90% confidence intervals for the comparison of the given formulation and the FORSTEO control for the ratios of Cₘₐₓ, AUCₗₐₛₜ and AUC_{inf} was calculated. The comparisons of each product with FORSTEO were done on a pairwise basis, but no adjustment for multiple testing was included because of the nature of this study.

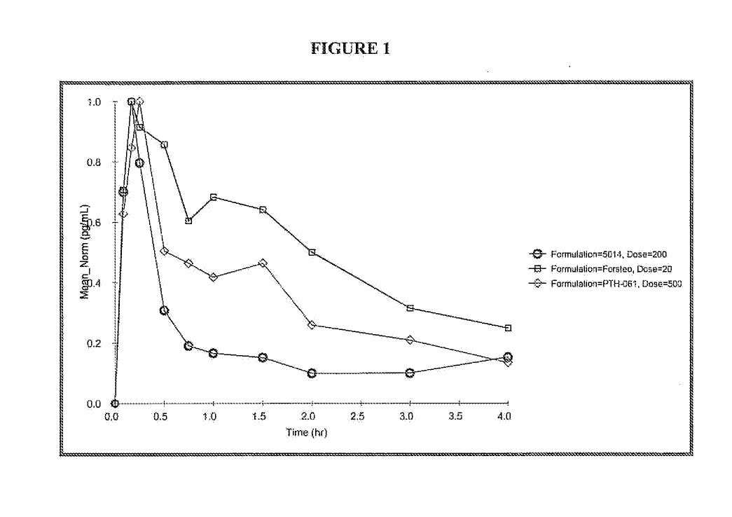

The pharmacokinetics is showing in graphic form in FIG. 1.

This graph shows the plasma levels of teriparatide following administration of three formulations, normalized to the Cₘₐₓ values for each. The graph compares two IN formulations (5014 and PTH-061) with a SC formulation (FORSTEO). As can be seen from the graph, FORSTEO administered subcutaneously has a longer period of elevated blood levels than either IN formulation. This result supports the conclusion that IN formulations can provide shorter periods of exposure to elevated blood levels of teriparatide. The shorter period of elevated therapeutic may provide an advantage in combination therapy with an antiresorptive.

A summary of clearance rates using the non-compartmental model are presented in Table 3:

TABLE 3
Summary of Clearance Rates

| Formulation | Dose (μg) | Mean (mL/hr) | SD |
|---|---|---|---|
| 5014 | 200 | 1366234.334 | 988398.4 |
| 5014 | 400 | 2527292.583 | 1701658 |
| FORSTEO | 20 | 267446.6298 | 263855.3 |
| PTH-061 | 500 | 4793716.136 | 4380229 |
| PTH-061 | 1000 | 3359436.634 | 1665618 |

A summary of percent coefficient of variation for each formulation and dose of teriparatide are presented in Table 4. Based on Cₘₐₓ and AUCₗₐₛₜ, the %CV is lower for formulation 05014 than formulation PTH-061 and FORSTEO.

TABLE 4
Percent Coefficient of Variation by Formulation and Dose

| | Dose | Tmax | Cmax | AUClast | AUCinf |
|---|---|---|---|---|---|
| Formulation | (ug) | (hr) | (pg/mL) | (hr*pg/mL) | (hr*pg/mL) |
| FORSTEO | 20 | 165.29 | 51.76 | 66.46 | 62.30 |
| PTH-061 | 500 | 142.48 | 78.71 | 92.76 | 83.41 |
| PTH-061 | 1000 | 176.56 | 67.06 | 75.55 | 71.56 |
| 05014 | 200 | 24.72 | 38.78 | 61.55 | 82.28 |
| 05014 | 400 | 21.20 | 48.78 | 55.98 | 68.04 |

A summary of percent relative bioavailability comparing each formulation to the FORSTEO product based on AUCₗₐₛₜ are presented in Table 5. The bioavailability of the 05014 formulation is 12-15%, whereas the PTH-061 is approximately 5-8 %.

TABLE 5
Relative Bioavailability Compared with
FORSTEO by Formulation and Dose

| Formulation | Dose (ug) | % Bioavailability |
|---|---|---|
| PTH-061 | 500 | 4.9 |
| PTH-061 | 1000 | 7.8 |
| 05014 | 200 | 14.6 |
| 05014 | 400 | 12.0 |

An exploratory compartmental analysis using WinNonLin 5.0 was conducted to compare the absorption coefficient and elimination coefficient for each formulation. A mixed model analysis of variance on both the Ka and the Ke data, where the subject was included as the random variable was performed, and these results were subanalyzed using the Tukey-Kramer multiple comparison procedure. The individual Ka and Ke data are presented in Table 6. The nasal absorption rates were not significantly different compared to FORSTEO (p=0.50), however the elimination rate for high dose nasal formulation 05014 was significantly faster (p=0.02) than FORSTEO. This is also observed when looking at the ratio of mean Cₘₐₓ to each individual time point per low dose formulation.

TABLE 6
Absorption Coefficient and Elimination
Coefficient for Each Formulation

| | | Dose | | Mean | | |
|---|---|---|---|---|---|---|
| Coefficient | Formulation | (μg) | N | (1/hr) | SD | CV % |
| Ka | FORSTEO | 20 | 11 | 11.99 | 7.00 | 58.34 |
| Ka | PTH-061 | 500 | 8 | 6.95 | 4.83 | 69.46 |
| Ka | PTH-061 | 1000 | 7 | 10.43 | 7.49 | 71.81 |
| Ka | 05014 | 200 | 6 | 11.02 | 5.29 | 48.05 |
| Ka | 05014 | 400 | 7 | 8.81 | 3.19 | 36.27 |
| Ke | FORSTEO | 20 | 11 | 1.04 | 0.86 | 83.50 |
| Ke | PTH-061 | 500 | 8 | 1.40 | 1.70 | 121.57 |
| Ke | PTH-061 | 1000 | 7 | 1.83 | 2.50 | 136.49 |
| Ke | 05014 | 200 | 6 | 2.74 | 2.24 | 81.85 |
| Ke | 05014 | 400 | 7 | 4.08 | 2.35 | 57.69 |

Based on the pharmacokinetic parameters, both nasal formulations had a greater Cₘₐₓ and AUC as compared to FORSTEO. The tₘₐₓ occurred sooner after dosing for the nasal formulations, particularly for the 05014 formulations. The absorption rates were not significantly different among the nasal and subcutaneous formulations (p=0.5), but elimination rates were faster particularly for the 05014 low dose nasal formulation (p=0.02).

However, a t_{1/2} of approximately 1 hour was very similar for the nasal formulations 10 compared to FORSTEO, except for the low dose 05014 formulation, where their maybe an apparent outlier for subject numbers 1 and 5. If the two subjects are removed the t_{1/2} is 1.5 hours, the same as FORSTEO. The apparent difference in elimination rates may reflect slower wash-in for the subcutaneous product and formulation PTH-061 when compared with the 05014 formulation.

Both nasal formulations have very similar t_{1/2} to FORSTEO. The nasal formulation 05014 also showed good dose linearity from 200 to 400 μg dose based on the clearance rate and regression analysis. In addition, the formulation was less variable than formulation PTH-061 and FORSTEO based on percent coefficient of variation. Accordingly, the intranasal formulations of the invention exceed the Cₘₐₓ and AUC values for the currently marketed subcutaneous product. This demonstrates that the levels of the marketed product can be exceeded by a nasally administered product, and also that the concentrations of PTH in nasal formulations can be decreased if it is desired to more closely approximate the plasma concentrations of the currently approved product.

What is claimed is:

1. A method for treating osteoporosis in a mammal comprising administering intranasally a therapeutically effective amount of a PTH formulation to the mammal in combination with administration of an anti-resorptive agent, wherein the PTH formulation contains a PTH peptide selected from the group consisting of SEQ ID NO: 1, SEQ ID NO: 2, and SEQ ID NO: 3.

2. The method of claim 1, wherein the PTH peptide has a Tmax, the time to maximum plasma concentration of the PTH peptide following administration of the PTH formulation, of less than 30 minutes.

3. The method of claim 1, wherein the PTH peptide has an elimination half life of about 55 minutes.

4. The method of claim 1, wherein the PTH peptide has a Cmax of greater than about 75 pg/ml.

5. The method of claim 4, wherein the Cmax is greater than about 150 pmole/l.

6. The method of claim 4, wherein the Cmax is greater than about 300 pg/ml.

7. The method of claim 1, wherein the PTH peptide has an AUClast greater than about 150 pg hr/ml.

8. The method of claim 1, wherein the PTH peptide is teriparatide (SEQ ID NO: 2).

9. The method of claim 1, wherein the PTH formulation is administered in parallel and simultaneously with the anti-resorptive.

10. The method of claim 1, wherein the PTH formulation is administered in a repeated series, comprising a first period wherein PTH is administered followed by a second period wherein anti-resorptive is administered.

11. The method of claim 10, wherein the first and second periods are each no greater than about twelve months.

12. The method of claim 10, wherein the first and second periods are each no greater than about six months.

13. The method of claim 1, wherein the anti-resorptive agent is a bisphosphonate.

14. The method of claim 13, wherein the bisphosphonate is selected from the group consisting of alendronate, raloxifene, ibandronate, risedronate, etridonate, clodronate, and mixtures thereof.

15. The method of claim 1, wherein the anti-resorptive agent is a hormone.

16. The method of claim 15, wherein the hormone is an estrogen or a progesterone.

17. The method of claim 1, wherein the anti-resorptive agent is a selective estrogen receptor modulator (SERM).

18. The method of claim 17, wherein the SERM is raloxifene.

19. A method for treating osteoporosis in a mammal comprising administering intranasally a therapeutically effective amount of a PTH formulation containing teriparatide to the mammal in combination with administration of a bisphosphonate.

20. The method of claim 19, wherein the PTH peptide has a Tmax of less than 30 minutes.

21. The method of claim 19, wherein the PTH peptide has a Cₘₐₓ of greater than about 75 pg/ml.
